(19) United States
(12) Patent Application Publication
(10) Pub. No.: US 2012/0220753 A1
(43) Pub. Date: Aug. 30, 2012

(54) N-Terminal Dimerization Methods with Bis-Amindino Acid and Bis-Thioimidate Derivatives

(76) Inventors: Lajos Gera, Denver, CO (US); Robert Hodges, Denver, CO (US); Richard C. Duke, Denver, CO (US)

(21) Appl. No.: 13/392,606

(22) PCT Filed: Aug. 26, 2010
(86) PCT No.: PCT/US10/46795

§ 371 Date: May 11, 2012

Related U.S. Application Data
(60) Provisional application No. 61/237,527, filed on Aug. 27, 2009.

(51) Int. Cl. C07K 1/04; C07K 1/00; C07C 327/42

(52) U.S. Cl. 530/334; 564/78; 530/333

(57) ABSTRACT

The invention provides high-yield protein dimerization methods using highly reactive bis-thioimidates that may be used in the manufacture of a highly potent anti-cancer peptide dimers.

### TECHNICAL FIELD

The present invention relates to processes for the dimerization of biologically active peptides using N-terminal dimerization methods with bis-amidino acid- and bis-thioimidate derivatives.

### BACKGROUND OF INVENTION

Bradykinin (BK) is a potent inflammatory peptide whose generation in tissues and body fluids elicits many physiological responses including vasodilation, smooth muscle spasm, edema, as well as pain and hyperalgesia (Burch et al., “Molecular Biology and Pharmacology of Bradykinin Receptors”, Landes Comp. (1993); Burch, edited: “Bradykinin Antagonists”, Dekker (1991)). There is increasing evidence that BK and related kinins contribute to the inflammatory response in acute and chronic diseases including allergic reactions, arthritis, asthma, sepsis, viral rhinitis, and inflammatory bowel disease. Recently BK was implied to be involved as an autocrine in the pathogenesis of human lung cancer (Bunn et al., Proc Natl. Acad. Sci. USA 87:2162-2166 (1990); Bunn et al., Cancer Research 52:24-31 (1992)). BK has been shown to be the most potent peptide stimulant of intracellular Ca⁺⁺ release in the highest fraction of human lung cancer cell lines (Bunn et al., Cancer Research 52:24-31 (1992)). The design and synthesis of specific, potent and stable bradykinin antagonists (BKA) has long been considered a desirable goal in medicinal chemistry. In the past few years, efforts have been directed towards the development of potent BK antagonists as a means for the chemoprevention and therapeutic treatment of human lung cancers.

Many lung and prostate cancers, of which small cell lung cancer (SCLC) is a prime example, have a neuroendocrine phenotype, and their growth is stimulated by neuropeptides. Antagonists of several peptides (e.g. bradykinin, substance P, bombesin) have been used in experimental treatment of models of SCLC in animals. Among the most potent of the peptides examined thus far, crosslinked dimers of certain bradykinin antagonist peptides have been efficacious both in vitro and in vivo against strains of SCLC and other tumors (Chan et al., Immunopharmacology 33: 201-204, 1996; Stewart et al., Can. J. Physiol. Pharmacol. 75: 719-724, 1997; Stewart et al., U.S. patent application Ser. No. 5,849,863, issued Dec. 15, 1998). Prostate cancers show a similar neuroendocrine phenotype and are susceptible to neuropeptide antagonists.

### SUMMARY OF INVENTION

The present invention provides a new bis-thio-imidate that can be used to dimerize any biologically active peptide at the N-terminal or through the amino-side chain with higher yield than with bis-imidates in current use. This serves to increase the peptide bioactivity or convert the peptide into an anti-cancer peptide dimer. A specific example of this dimerization resulting in a potent anti-cancer peptide dimmer is in the production of the compound B9870.

### DESCRIPTION OF EMBODIMENTS

The present invention is exemplified by the following description of the enhanced production of the compound B9870, and no limitation as to the scope of the present invention is intended by either the inclusion or non-inclusion of elements, components, etc. in the use of this protein compound as an example of the present invention. Additional aspects of the present invention will become more readily apparent from the details of the synthesis of the B9870 compound.

B9870 (also known as B201, CU201, NSC 710295, Breceptin) was designed, developed, synthesized and its effects discovered more than a decade ago as a highly potent anti-cancer bradykinin (BK) antagonist peptide bis-amidine dimer (Gera, Lajos; Stewart, John M.; Whalley, Eric; Burkard, Michael; Zuzack, John S. A new class of potent bradykinin antagonist dimers. Immunopharmacology (1996), 33(1-3), 178-182; Chan, Daniel; Gera, Lajos; Helfrich, Barbara; Helm, Karen; Stewart, John; Whalley, Eric; Bunn, Paul. Novel bradykinin antagonist dimers for the treatment of human lung cancers. Immunopharmacology (1996), 33(1-3), 201-204; Stewart, John M.; Chan, Daniel C.; Whalley, Eric T.; Gera, Lajos. Cytolitic bradykinin antagonists. U.S. Pat. No. 5,849,863, 15 Dec. 1998; Chan, Daniel; Gera, Lajos; Stewart, John; Helfrich, Barbara; Verella-Garcia, Marileila; Johnson, Gary; Baron, Anna; Yang, Jie; Puck, Theodore; Bunn, Paul, Jr. Bradykinin antagonist dimer, CU201, inhibits the growth of human lung cancer cell lines by a “biased agonist” mechanism. Proceedings of the National Academy of Sciences of the United States of America (2002), 99(7), 4608-4613; Feng, Wan Yong; Chan, Kenneth K.; Covey, Joseph M. Electrospray LC-MS/MS quantitation, stability, and preliminary pharmacokinetics of bradykinin antagonist polypeptide B201 (NSC 710295) in the mouse. Journal of Pharmaceutical and Biomedical Analysis (2002), 28(3-4), 601-612.

The enzyme-resistant, highly potent BK B1/B2 receptor antagonist peptide B9430, was previously developed using solid phase-peptide synthesis (Gera, Lajos; Stewart, John M. A new class of bradykinin antagonists containing indanylglycine. Immunopharmacology (1996), 33(1-3), 174-177) and was dimerized to B9870 by a conventional cross-linking reagent such as suberimidate at the N-terminal. The cross-linking procedure for purified B9430 was carried out in solution which resulted in a moderate yield of less than 40% (Gera, Lajos; Stewart, John M.; Whalley, Eric; Burkard, Michael; Zuzack, John S. A new class of potent bradykinin antagonist dimers. Immunopharmacology (1996), 33(1-3), 178-182). The limited yield by this procedure thus potentially decreases its commercial value. Imidates can form amidines with amino acids with excellent yield at elevated temperature (Bernath, Gabor; Toth, Gabor; Fulop, Ferenc; Gondos, Gyorgy; Geral Lajos. Saturated heterocycles. Part 7. Stereochemical studies. Part 30. Synthesis and conformational analysis of deca-and dodecahydropyrido[2,1-b]quinazolin-11-ones. Journal of the Chemical Society, Perkin Transactions 1 (1979), 7, 1765-1769) but these reaction conditions are not recommended for temperature sensitive peptides.

A preferred method of producing a high yield of B9870 (Breceptin), a potent anti-cancer drug candidate, is by using N-terminal dimerization methods with bis-amidino acid and bis-thioimidate derivatives. This high yield can be greater than 85%.

A preferred embodiment of the present invention is improved processes for the manufacture of compound B9870, at a commercial scale.

In yet another aspect, the present invention provides processes of making compound B9870 with increased overall yield by using suberyl-thioimidate reagents to achieve the desired amidine-dimerization process of B9870 dimer, which can increase the commercialization value of B9870 by decreasing the cost of the synthesis.

Another aspect of the invention combines the process of dimerization on a solid phase without monomer isolation and purification steps with the process of using bis-amidino acid derivatives or suberyl-thioimidate reagents to achieve high yield synthesis of B9870.

The new high-yield dimerization methods of the present invention with highly reactive bis-thioimidates can replace the bis-imidates in bioconjugate crosslinking techniques, for example in peptide-, protein- and medicinal research since they are more reactive than the bis-imidates. The bis-amidino acid derivatives and the bis-thioimidates can be prepared by the general methods (Shearer, Barry G.; Oplinger, Jeffrey A.; Lee, Shuliang. S-2-Naphthylmethyl thioacetimidate hydrobromide: a new odorless reagent for the mild synthesis of substituted acetamidines. Tetrahedron Letters (1997), 38(2), 179-182; Hansen, Marvin M.; Borders, Sandra S. K.; Clayton, Marcella T.; Heath, Perry C.; Kolis, Stanley P.; Larsen, Samuel D.; Linder, Ryan J.; Reutzel-Edens, Susan M.; Smith, Justin C.; Tameze, Shella L.; Ward, Jeffrey A.; Weigel, Leland O. Development of a practical synthesis of an aminoindanol-derived M1 agonist. Organic Process Research & Development (2009), 13(2), 198-208; Hoffmann, Rainer; Hartke, Klaus. Thiono and dithio esters, XX. Dithiono and tetrathio esters of higher dicarboxylic acids. Justus Leibigs Annalen der Chemie (1977), (10), 1743-50; Hammond, Ming C.; Bartlett, Paul A. Synthesis of amino acid-derived Cyclic acyl amidines for use in β-strand peptidomimetics. The Journal of Organic Chemistry (2007), 72(8), 3104-07; Guzman, Angel; Romero, Moises; Talamas, Francisco X.; Villena, Rene; Greenhouse, Robert; Muchowski, Joseph M. 1,3-Diaza-1,3-butadienes. Synthesis and Conversion into Pyrimidines by [4π+2π] Cycloaddition with Electron Deficient Acetylenes. Synthetic Utility of 2-(Trichloromethyl)pyrimidines. The Journal of Organic Chemistry (1996), 61(7), 2470-83).

In one embodiment, the present invention provides processes for the preparation of the compound B9870. The compound of B9870 (Formula IV) can be prepared for example, by the following reaction sequence as depicted in Scheme I below:

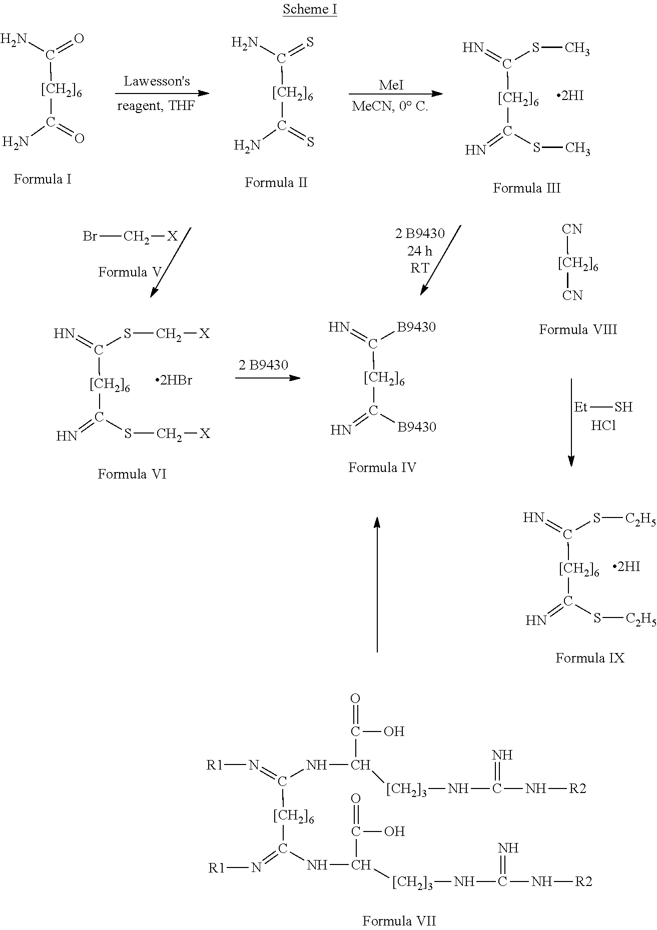

B9870 may be represented by the following formula:

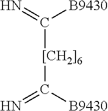

Wherein B9430 is the bradykinin antagonist monomer:
DArg-Arg-Pro-Hyp-Gly-Igl-Ser-DIgl-Oic-Arg
   wherein Hyp represents trans-4-Hydroxy-Pro; Igl represents α-(2-Indanyl)glycine; and Oic represents Octahydroindole-2-carboxylic acid. As used herein, abbreviations of the natural amino acids are those accepted in the art (Biochem. J. 126:773 (1972)), and unless prefixed with a D are all of the L-configuration.

The linker, SUIM (suberimidyl), is a cross-linking reagent that is produced by reacting suberamide (octanediamide) (Formula I) with a thionating agent, such as Lawesson's reagent in tetrahyrofuran (THF) resulting in octaneditioamide (Formula II). Formula II is converted into Formula IV in two ways:
i) by reacting Formula II with methyl iodide (MeI) in acetonitrile (MeCN) resulting in bis-thioamidate (which has enhanced leaving group reactivity compared to alcohols, Formula III) which is then reacted with DArg-Arg-Pro-Hyp-Gly-Igl-Ser-DIgl-Oic-Arg resulting in Formula IV, or
ii) by reacting Formula II with Formula V, Br-CH₂-X (wherein X is H, alkyl, aryl, heteroalkyl, cycloalkyl or 2-naphtyl) resulting in Formula VI which is then reacted with DArg-Arg-Pro-Hyp-Gly-Igl-Ser-DIgl-Oic-Arg resulting in Formula IV.

With regard to the linker length, the linker may comprise alkyl chains of 0 carbons in length or greater. Alkyl chains of 6 carbons are preferred. Chain lengths of 6 to 20 carbons may also be used.

In addition, Formula VII (wherein R1 is H or Boc, Fmoc and R2 is Tos, Mts, Mtr, Pmc or Pbf) can be coupled with Arg-Pro-Hyp-Gly-Igl-Ser-DIgl-Oic-Arg resulting in Formula IV. In this process the process proceeds on a solid phase without monomer isolation and without purification. Boc represents tert-Butoxycarbonyl; Fmoc represents Fluorenylmethoxycarbonyl; Tos represents p-Toluenesulfonyl; Mts represents 2-mesitylenesulfonyl; Mtr represents 4-methoxy-2,3,6-trimethylphenylsulfonyl; Pmc represents 2,2,5,7,8-pentamethylchroman, and Pbf represents 2,2,4,6,7-pentamethyldihydrobenzofurane.

The monomers (B9430) may be linked via the N-terminus, either through the terminal arginine or through an added lysine residue. It is preferred that there is at least one basic charge at the amino end of the dimerized or monomer-linker compound. For example, the charge may be on the amino group of an N-terminal lysine residue or on the imide group of the linker.

In the above reaction scheme, where specific reducing agents, solvents, bases, catalysts, acids etc., are mentioned, it is to be understood that other reducing agents, solvents, bases, catalysts, acids etc., known to those skilled in the art may be used. Similarly, the reaction temperature and duration may be adjusted.

While the present invention has been described in terms of its specific embodiments, certain modifications and equivalents will be apparent to those skilled in the art and are included within the scope of the present invention.

### EXAMPLES

### Example 1

### Preparation of B9870 (Formula IV) via Formula V:

To a solution of suberamide (octanediamide) (Formula I) was added Lawesson' reagent in tetrahyrofuran (THF) resulting in octaneditioamide (Formula II). Formula II is reacted with methyl iodide (MeI) in acetonitrile (MeCN) at 0° C. resulting in Formula III. Formula III is then reacted with 2 equivalents of DArg-Arg-Pro-Hyp-Gly-Igl-Ser-DIgl-Oic-Arg for 24 hours which results in a high yield of Formula IV.

### Example 2

### Preparation of B9870 (Formula IV) via Formula III:

To a solution of suberamide (octanediamide) (Formula I) was added Lawesson' reagent in tetrahyrofuran (THF) resulting in bis-thioamide (Formula II). Formula II is reacted with Formula V, Br-CH₂-X (wherein X is H, alkyl, aryl, heteroalkyl, cycloalkyl or 2-naphtyl) resulting in Formula VI which is then reacted with 2 equivalents of DArg-Arg-Pro-Hyp-Gly-Igl-Ser-DIgl-Oic-Arg resulting in a high yield of Formula IV.

### Example 3

### Preparation of B9870 (Formula IV) via Formula VII:

A solution of Formula VII is coupled with Arg-Pro-Hyp-Gly-Igl-Ser-DIgl-Oic-Arg side chain protected peptide on a solid phase without monomer isolation and without purification, resulting in a high yield of Formula IV.

What is claimed is:

1. A method of making Formula II, 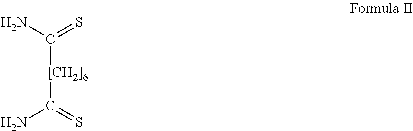
the method comprising reacting Formula I with a thionating agent in tetrahydrofuran, 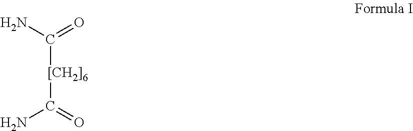
to form a compound of Formula II.

2. A method of making Formula IV, 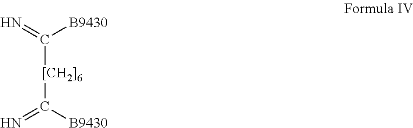
the method comprising:
(a) reacting a compound of Formula II with methyl iodide in acetonitrile, to form a compound of Formula III; 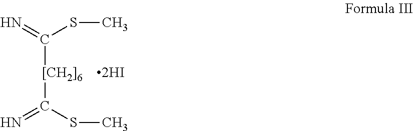
(b) reacting a compound of Formula III with DArg-Arg-Pro-Hyp-Gly-Igl-Ser-DIgl-Oic-Arg, to form a compound of Formula IV.

3. A method of making Formula IV, 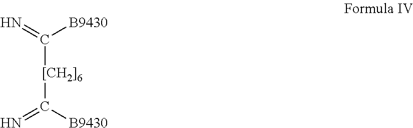
the method comprising:
(a) reacting a compound of Formula II with a compound of Formula V wherein X is H, alkyl, aryl, heteroalkyl, cycloalkyl or 2-naphtyl,
Br-CH₂-X Formula V
to form a compound of Formula VI, 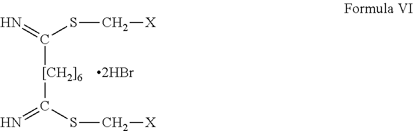
(b) reacting a compound of Formula VI with DArg-Arg-Pro-Hyp-Gly-Igl-Ser-DIgl-Oic-Arg, to form a compound of Formula IV.

4. A method of making Formula IV, 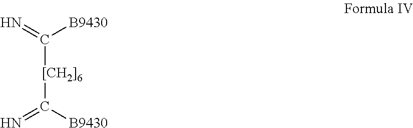
the method comprising coupling a compound of Formula VII wherein R1 is H, Boc or Fmoc and wherein R2 is Tos, Mts, Mtr, Pmc or Pbf with Arg-Pro-Hyp-Gly-IGl-Ser-DIgl-Oic-Arg side chain protected peptide on a solid phase: 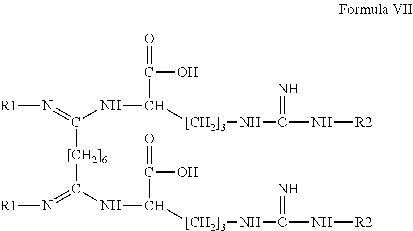
to form a compound of Formula IV.

5. A method of making Formula VI, 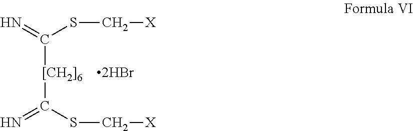
the method comprising reacting a compound of Formula II with a compound of Formula V wherein X is H, alkyl, aryl, heteroalkyl, cycloalkyl or 2-naphtyl, 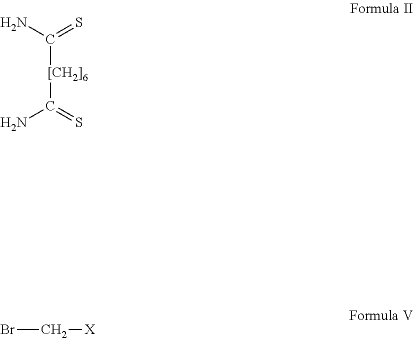
to form a compound of Formula VI.

6. The process according to claim 1, wherein the thionating agent is Lawesson's reagent.

7. The process according to claim 2, wherein the reaction step of (a) is carried out at a temperature between about 0° C. and about 80° C.

8. The process according to claim 2, wherein the reaction step of (b) is carried out at a temperature between about 0° C. and about 80° C. for about 24 hours.

9. The process according to claim 4, wherein the process proceeds on a solid phase without monomer isolation and purification.

10. A method of making Formula IV, 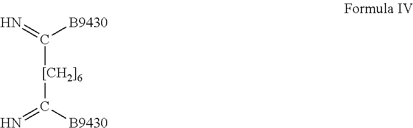
the method comprising contacting a suberonitrile compound of Formula VIII with Et-SH and HCl: 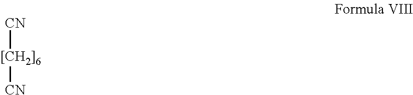
to form a compound of Formula IX: 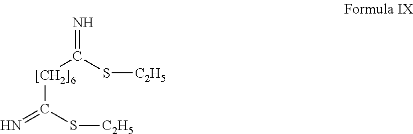
Contacting a compound of Formula IX with 2 equivalent of B9430 (DArg-Arg-Pro-Hyp-Gly-Igl-Ser-DIgl-Oic-Arg) to form a compound of Formula IV.
